(19) United States
(12) Patent Application Publication
(10) Pub. No.: US 2023/0201007 A1
(43) Pub. Date: Jun. 29, 2023

(54) COSMESIS, SYSTEM CONSISTING OF PROSTHETIC COMPONENT AND COSMESIS, AND CONNECTION PART

(71) Applicant: OTTO BOCK HEALTHCARE PRODUCTS GMBH, Wien (AT)

(72) Inventors: Johannes BISCHOF, Wiener Neustadt (AT); Sonja WAGNER, Wien (AT)

(21) Appl. No.: 17/999,669

(22) PCT Filed: May 27, 2021
(86) PCT No.: PCT/EP2021/064224

§ 371 Date: Nov. 22, 2022

(30) Foreign Application Priority Data
May 29, 2020 (DE) 10 2020 114 497.0

(51) Int. Cl. A61F 2/60; A61F 2/70

(52) U.S. Cl. CPC A61F 2/60; A61F 2/70; A61F 2002/5001

(57) ABSTRACT

The invention relates to a prosthetic cosmetic cover (1) having a base body (11) which forms a cavity (12) for at least partially receiving a prosthetic component (3) and has an inner side (13) facing the prosthetic component (3) and an outer side (14) facing away from the prosthetic component (3), wherein at least one first electrical connection element (21) is arranged on the inner side (13) and at least one second electrical connection element (22) is arranged on the outer side (14), which are electrically connected to one another.

The present invention relates to a prosthetic cosmetic cover having a base body which forms a cavity for at least partially receiving a prosthetic component and has an inner side facing a prosthetic component and an outer side facing away from the prosthetic component. The invention also relates to a system comprising a prosthetic component and such a prosthetic cosmetic cover, and to a connection part as such.

Prosthetic cosmetic covers as well as protectors fulfill on the one hand a protective function to protect the prosthetic component arranged in a cavity of the cosmetic cover from external influences, and on the other hand serve to make the prosthetic component visually and haptically pleasant. Prosthetic cosmetic covers therefore usually approximate the shape of the natural limb.

To provide cushioning for the prosthetic component arranged in the prosthetic cosmetic cover, prosthetic cosmetic covers often have a base body made of foam.

Prosthetic devices that are covered with prosthetic cosmetic covers may be arranged on an upper or lower limb. The prosthetic devices may include electrical and electronic components that are used to activate and deactivate drives, or that record sensor data and modify it based on programs to perform specific controls in drives or damping devices. For this purpose, data processing systems are assigned to the prosthetic devices, in particular integrated therein.

In order to maintain the function of such data processing systems, power must be supplied to the system and/or there must be a way of transmitting external data. Furthermore, power must be supplied to drives or adjustment devices. The systems therefore feature interfaces by which, for example, an accumulator can be charged or data can be transferred.

Power is supplied to a data processing system arranged in the prosthetic component or the energy supply system, for example, by connecting a charging cable or data transmission cable to an interface.

For this purpose, it is often necessary to remove the prosthetic cosmetic cover and/or to remove the data processing system or the energy supply system or the accumulator from the prosthetic component. In these cases, handling is more difficult for the prosthesis user; the user may need to rely on the assistance of an orthotist. If possible, accessibility to the interface should always be ensured without damaging or compromising the prosthetic cosmetic cover. Prosthetic cosmetic covers cannot normally be removed easily; rather, special tools are required. In order to enable access to the interface, holes are sometimes subsequently made in the prosthetic cosmetic cover, thus damaging the prosthetic cosmetic cover.

If the interface is attached to the outer side of the prosthetic component, the interface can be concealed by a prosthetic cosmetic cover subsequently placed over it. If, on the other hand, a recess is formed in the prosthetic cosmetic cover through which the interface can be reached, for example via a connector, the external appearance is impaired.

The invention is therefore based on the task of providing a prosthetic cosmetic cover which overcomes or at least reduces the aforementioned disadvantages and enables simplified handling for the user.

This task is solved by a prosthetic cosmetic cover with the features of the main claim as well as a system and a connection part with the features of the respective independent claim. Further embodiments and advantageous embodiments of the inventions are disclosed in the subclaims, the description and the figures.

The prosthetic cosmetic cover according to the invention provides that a base body of the prosthetic cosmetic cover for at least partially receiving the prosthetic component forms a cavity and has an inner side facing the prosthetic component and an outer side facing away from the prosthetic component, wherein at least one first electrical connection element is arranged on the inner side and at least one second electrical connection element is arranged on the outer side, which are electrically connected to one another. The at least one first connection element arranged on the inner side of the prosthetic cosmetic cover can be connected to an interface of the data processing system arranged in the prosthetic component and/or of the power supply system. The electrical connection between the at least two connection elements arranged on the cosmetic cover enables the user to supply power to the data processing system and/or the power supply system without having to remove the prosthetic cosmetic cover and/or impair its external appearance.

In one embodiment, the electrical connection between the at least two connection elements comprises an electrical conductor. This ensures a secure and permanent electrical connection between the at least two connection elements, whereby the positioning of the connection elements on the prosthetic cosmetic cover or the base body can be flexibly adapted to the needs of the user during production.

The electrical conductor can be formed as a textile conductor track, a conductive yarn, wire or the like. Likewise, the conductor may have an insulating layer, for example made of plastic.

The connection elements may be designed as a socket, a contact field and/or a plug. In one embodiment, the at least one first connection element is designed as a plug to be connected to an interface designed as a socket on the data processing system and/or the power supply system. On the other hand, the second connection element can be designed as a socket, so that the interface provided on the data processing system and/or the power supply system is doubled on the outer side of the cosmetic cover or routed thereto. This offers the advantage that the user can establish a connection to the electrical and/or electronic components of the prosthetic component without having to remove the data processing system and/or the power supply system or having to remove the prosthetic cosmetic cover.

A further embodiment of the connection elements provides that they are designed as contact fields or plugs to enable data and/or power transmission.

In the embodiment as socket, plug and/or contact field, the connection elements can have a magnetic securing system. While in the connection between a plug and a socket at least one form-fit component is provided, a magnetic securing system leads to a force-fit component. In particular, in the case of a contact field, a magnetic securing system secures the contact between the connection elements and the respective mating part. If an external source is connected to such a connection element by means of a cable connection, a risk of falling due to entanglement in the cable connection is at least reduced, since the contacting is released in such a case.

The base body can be designed in multiple parts. In particular, a modular design of the components of a base body makes it possible to manufacture components of the prosthetic cosmetic cover in series and to provide a final version adapted to the user at low cost.

In this context, at least one of the parts forming the base body of the prosthetic cosmetic cover can be made from a foam material. On the one hand, foam enables prosthetic cosmetic covers to be designed with a low weight. On the other hand, it provides effective cushioning of the prosthetic part and therefore protects not only the prosthetic part from external influences, but also the user of the prosthetic part.

In a multi-part embodiment, a connection part may be arranged at one end of the base body, which forms part of the base body and has an inner side facing the prosthetic component and an outer side facing away from the prosthetic component. The connection part may form a terminal edge of the prosthetic cosmetic cover.

The connection part may further serve to form the connection to another prosthetic component and/or prosthetic cosmetic cover component. It is conceivable, for example, that a prosthetic limb is at least partially covered by the prosthetic cosmetic cover according to the invention. The connection part then forms the connection to a joint section of the prosthesis at a proximal and/or distal end of the base body.

In the case of a multi-part construction of the base body, the connection part may be made from a more rigid material than the material of the rest of the base body. The connection part may be made of a plastic and manufactured in processes commonly used for such parts, such as an injection molding process or an additive manufacturing process. The rigid design of the connection part ensures sufficient stability to provide a stable assignment of the connections for the connection elements. On the one hand, the connection part serves as a support for the connection elements and, on the other hand, as part of the prosthesis cosmetic cover for shaping. The connection part completes the prosthetic cosmetic cover on the base body to create the final shape. Advantageously, a flush transition between the base body and the connection part is achieved during or after assembly of the connection part to the base body. Despite the more rigid embodiment, the connection part may have a certain flexibility and elasticity so that the connection part can be deformed when a deforming force is applied and, when the deforming force is removed by restoring forces, the connection part returns to its original shape in a non-destructive manner.

A further embodiment of the invention provides that the connection elements provided for supplying power to a data processing system and/or a power supply system are arranged on the connection part, the first connection element being arranged on the inner side of the connection part and/or the at least one second connection element being arranged on the outer side of the connection part.

The electrical connection between two connection elements may comprise an electrical conductor arranged in the connection part. For example, the conductor has been incorporated into the connection part during manufacturing, for example inserted into the mold during an injection molding process or additive manufacturing and enclosed or at least partially surrounded by the material. The electrical conductor may also be secured within the connector part in one or more mounts, wherein the mounts may be integrally formed with the connection part. The conductor with the connection elements is then subsequently fixed to the connection part. The electrical conductor can be arranged in a duct in the connection part and/or the base body. This makes it possible to arrange the electrical conductor on or in the connection part and/or on or in the base body after the connection part has been manufactured. A duct can be formed, for example, as a hole and/or recess made in the material of the base body or as a channel, so that the electrical conductor is fixed to the inner side of the prosthetic cosmetic cover, protrudes to only a small extent or not at all, and does not come into contact with the prosthetic component. The conductor may be integrated into the base body.

The electrical conductor may be arranged with or without an insulating layer in or on the connection part and/or in or on the base body.

By electrically connecting the two connection elements with a conductor, the positioning of the interface to the prosthetic component can be flexibly selected during the manufacture of the prosthetic component, since accessibility is determined by the positioning of the respective connection elements on the prosthetic cosmetic cover. The patient's access to the interface for the first connection element, which is arranged on the inner side of the connection component, is irrelevant; it is only necessary that a connection to the interface arranged on the prosthetic component can be made when the prosthetic cosmetic cover is put on. Easy access for the user is achieved by the second electrical connection element arranged on the outer side, which is appropriately positioned on the prosthetic cosmetic cover to facilitate connection of an external power source and/or data source.

In one embodiment, the first connection element is fixed in a form-fitting manner to the connection part. This can be done by incorporating the connection element during manufacture of the connection part, so that it is cast, for example. However, it is also conceivable that a suitable recess is drilled, milled or made in another suitable way in the connection part, with which the connection element can be engaged in a form-fitting manner. The form-fit ensures a stable hold of the connection element.

In particular, the connection part can be fixed to the base body of the prosthetic cosmetic cover in a form-fitting or material-bonded manner. It is conceivable, for example, that the connection part is foamed or glued into the base body. By making the connection part from a more rigid material than the rest of the base body, the cosmetic cover has a stiffened area, for example a stiffened end area, which enables easy connection of a plug or a connection element. In combination with the permanent connection of the connection part to the base body, which is ensured by form-fit or material bonding, the handling of the prosthetic cosmetic cover is made easier.

A further embodiment of the invention provides that the connection part is designed in multiple parts. The multi-part design of the connection part facilitates assembly. In addition, replacement parts can be replaced more easily.

The connection part can have at least one fastening element for fastening to the prosthetic component. Via the fastening element, the connection part is fixed to the prosthetic component; advantageously, it is fixed in such a way that the connection part can be detachably removed again without destroying it. Preferably, a mechanical fastening of the connection part to the prosthetic component is provided, for example via projections and undercuts, clips, clamps, hooks or the like. The connection part is directly fixed to the prosthetic component via the fastening element, whereby an improved connection of the base body of the prosthetic cosmetic cover to the prosthetic component can also be achieved.

The connection part is advantageously fixed to the base body of the prosthetic cosmetic cover such that it can be removed, so that it can be replaced without destroying the base body and the connection part. The fastening is done, for example, by pinning, clamping, undercuts, clips or separate fastening elements. It is also possible to attach the connection part to the base body such that it can be removed using a Velcro fastener. The connection part can be arranged at least partially on the inner side of the base body, so that the base body partially covers the outer side of the connection part and, if necessary, clamps it to the prosthetic component.

The connection elements may be integrated into the base body of the prosthetic cosmetic cover. Likewise, ducts or accommodations for an electrical conductor may be formed or integrated within the base body. Holders for the respective connection elements may be arranged or formed on the base body and/or the connection part, which allow the connection elements to be fixed in a variable position. For example, rails or rail-like accommodations or ducts can be arranged or formed within the base body and/or connection part, along which the connection element or elements can be movably mounted and fixed in the desired position. They can be fixed by means of form-fitting elements or by clamping or latching in the respective desired position.

Subject matter of the invention is also to provide a system comprising a prosthesis or prosthetic component and a prosthetic cosmetic cover having the features described above. In this case, the prosthetic cosmetic cover accommodates the prosthetic component at least partially in a cavity provided for this purpose. The cavity is adapted to the outer contour of the prosthesis or prosthetic component. The prosthetic cosmetic cover is provided with fastening elements or accommodations for the prosthetic component or the prosthesis, so that the prosthetic cosmetic cover can be fixed to the prosthesis or prosthetic component. In particular, the outer shape of the prosthetic cosmetic cover is adapted or approximated to the contour of the limb on the contralateral side or is adapted or approximated to a contour of the limb to be replaced.

A data processing system and/or a power supply system may be arranged in the prosthesis or prosthetic component, and connected to the at least one first connection element on the inner side of the prosthetic cosmetic cover.

An electrical interface is arranged on the data processing system and/or the power supply system, which can be electrically connected to the at least one first connection element. The electrical connection between the connection elements arranged on the prosthetic cosmetic cover makes it possible to supply the data processing system and/or the power supply system with an external source without having to remove the prosthetic cosmetic cover or take out the systems to be supplied.

According to the invention, the prosthetic cosmetic cover may be configured with a connection part on which the connection elements are arranged as previously described.

Particularly preferably, the at least one first connection element is arranged on the inner side of the connection part in such a way that the connection element can be electrically connected to the interface of the data processing system and/or the power supply system by applying the prosthetic cosmetic cover. Advantageously, the interface and the connection element are positioned corresponding to each other so that they make direct contact when the prosthetic cosmetic cover is applied. The design of the interface and connection element is freely selectable. When designed as socket and plug, the connection is secured by a form-fit component. When designed as a contact field, in particular with magnetic securing, a force closure component is provided.

The system of prosthetic component and prosthetic cosmetic cover according to the invention further provides that the prosthetic cosmetic cover is detachably arranged on the prosthetic component. This ensures free access to the prosthesis arranged in the cosmetic cover, in particular in the event of a malfunction. The user of the prosthesis is also free to choose the prosthetic cosmetic cover to be applied.

As a component of a multi-part base body of a prosthetic cosmetic cover with an inner side facing the prosthetic component and an outer side facing away from the prosthetic component, a connection part in the sense of the present invention is characterized in that at least one first electrical connection element is arranged on the inner side and at least one second electrical connection element is arranged on the outer side, which are electrically connected to one another.

Examples of embodiments of the invention are explained in more detail below with reference to the figures. They show:

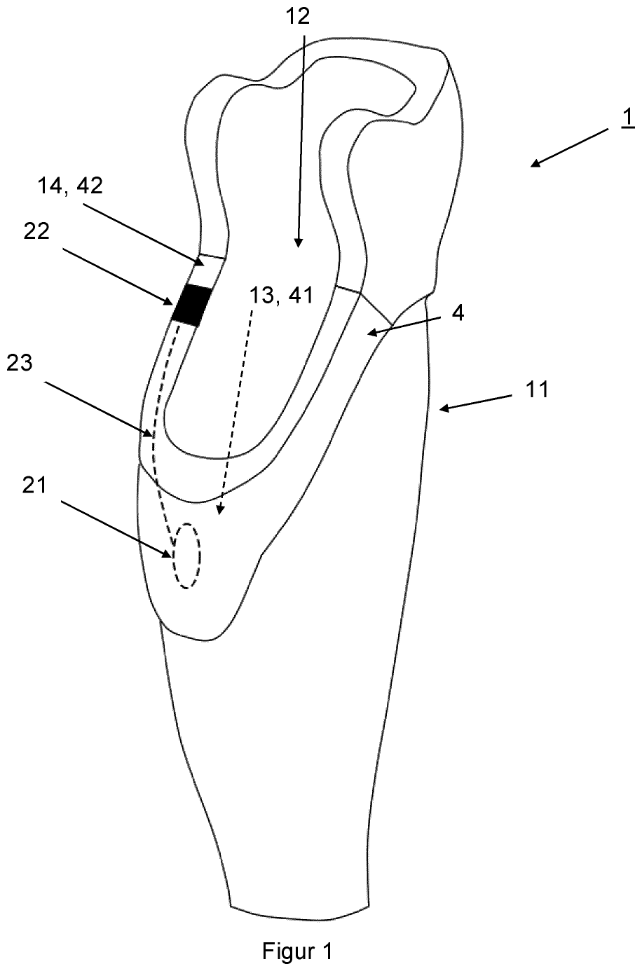

FIG. 1—a schematic representation of a prosthetic cosmetic cover with a multi-part base body;

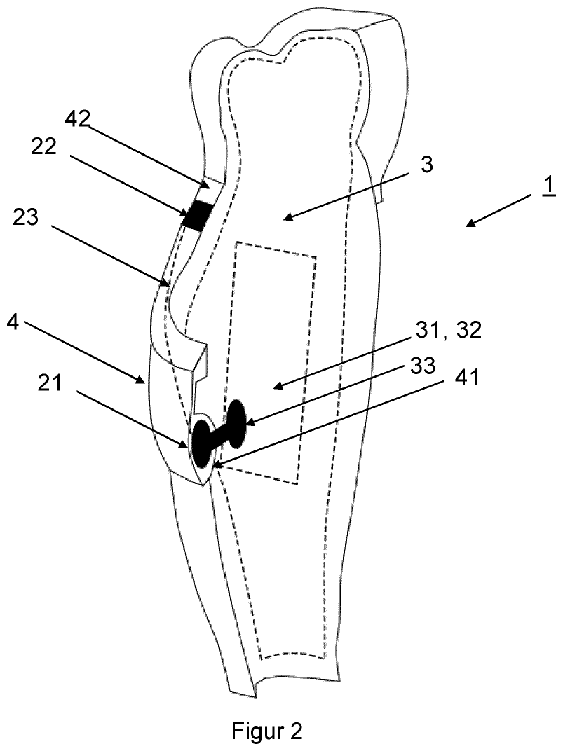

FIG. 2—a sectional view through a prosthetic cosmetic cover with schematically shown prosthesis;

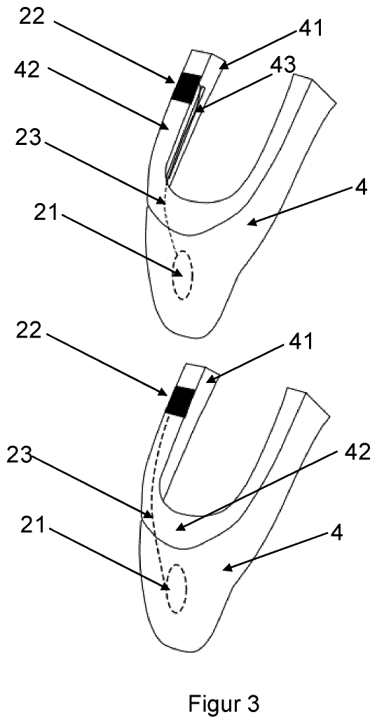

FIG. 3—schematic representations of a connection part with two variants of conductor routing; and

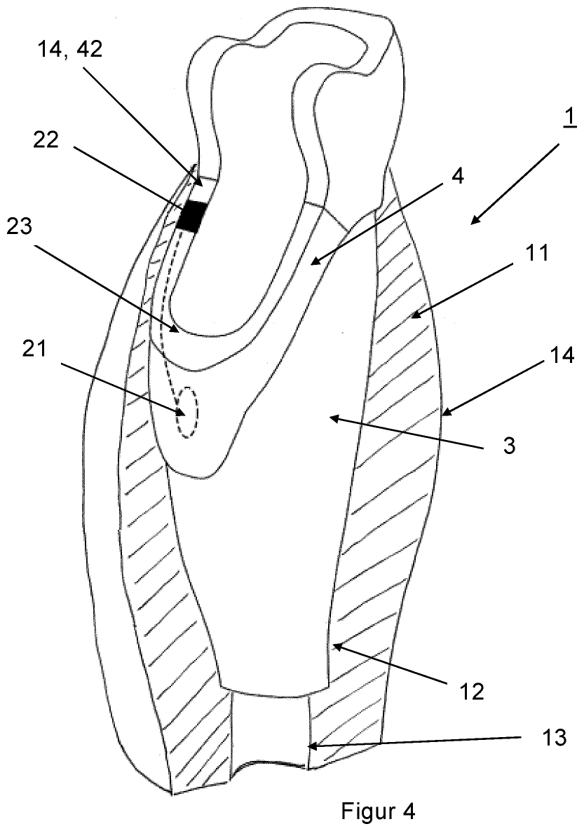

FIG. 4—a variant with a partially embedded connection part.

FIG. 1 shows a schematic representation of a prosthetic cosmetic cover 1 with ax\hat{ }\hat{ } base body 11, which has a cavity 12 for at least partially accommodating a prosthetic component 3 shown in FIG. 2. An inner side 13 of the base body 11 faces the prosthetic component 3, and an outer side 14 faces away from the prosthetic component 3. All the sides that point away from the prosthetic component 3 or point past the prosthetic component are to be understood as facing away from the prosthetic component 3, in particular also upper sides of end edges of the base body 11.

In the embodiment according to FIG. 1, a lower leg prosthetic cosmetic cover is shown. A lower leg prosthesis, at the proximal end of which a prosthetic knee joint is connected, can be at least partially accommodated in the cavity 12. At a proximal end, the base body 11 is provided with a connection part 4, which forms the termination of the proximal end of the base body 11 in the form of a closed contour.

As a component of the base body 11, the connection part 4 has an inner side 41 facing the prosthetic component 3 and an outer side 42 facing away from the prosthetic component. The upper edge of the connection part 4 forms an outer side 42.

The connection part 4 may be formed of a more rigid material than the material of the rest of the base body 11. For the distal portion of the base body 11, which in the exemplary embodiment covers the prosthetic component 3 and has the shape of a lower leg, foams have proven useful, for example. Foams have a low density, so that a design of components of the base body 11 made of a foam ensures the provision of a light prosthetic cosmetic cover. The connection part 4 can be made of a plastic which has a higher rigidity than the foam of the distal part of the base body 11. The connection part 4 may be attached to the proximal end of the base body 11 in a form-fitting and/or material-bonded manner. For example, the connection part 4 can be foamed in during production of the remaining base body 11 from a foam material. It is also conceivable to glue the connection part 4 to the proximal end of the base body or to connect it in another suitable manner by way of material bonding. Alternatively, the connection part 4 is placed or plugged onto the rest of the base body 11 and clamped in place or fixed by form-fit elements.

The connection part 4 can be brought into form-fitting engagement with a section of a prosthetic component 3. Projections or recesses on the inner side 41 of the connection part 4 can engage with corresponding recesses or projections on the prosthetic component 3.

In the illustrated embodiment example, electrical connection elements 21, 22 are arranged in the connection part 4. Here, a first connection element 21 is arranged on an inner side 41 of the connection part 4 facing the prosthetic component 3 or the cavity 12. The second connection element 22 is arranged on an outer side 42 facing away from the prosthetic component 3 or the cavity 12. There is an electrical connection between the two connection elements 21, 22, exemplarily shown as an electrical conductor 23.

The first connection element 21 can be fixed in the connection part 4 in a form-fitting manner. This can be done by incorporating the connection element 21 during production of the connection part 4, so that it is cast in, for example. However, it is also conceivable that a suitable recess is drilled, milled or otherwise made in the connection part 4, with which the connection element 21 can be engaged in a form-fitting manner.

The electrical connection elements 21, 22 can be in the form of a plug, socket and/or electrical contact field. The electrical connection between the connection elements 21, 22 can be designed as an electrical conductor 23.

Insofar as the respective connection element 21, 22 is designed as an electrical contact field, it can have a magnetic securing. By means of a magnetic securing, a force-fit component acts on the contacting to ensure the hold.

In the embodiment shown, the electrical conductor 23 is arranged in the connection part 4 and is formed as an insulated cable; other embodiments are possible, for example printed conductor tracks, electrically conductive plastics or textiles or other signal and current conducting materials and components.

FIG. 2 shows a sectional view through a prosthetic cosmetic cover 1 with schematically shown prosthetic component 3. A data processing system 31 and/or a power supply system 32 is arranged inside the prosthetic component 3 that has an interface 33 for connection to an external source not shown.

In the illustrated embodiment, the first connection element 21 is arranged on the inner side 41 of the connection part 4. The form-fitting fixture of the connection element 21 in the connection part 4 ensures the secure hold of the connection element 21. The connection element 21 and the interface 33 are arranged in a corresponding manner, so that when the prosthetic cosmetic cover 1 is applied, the connection element 21 and the interface 33 can be directly electrically connected.

The connection elements 21, 22 are electrically connected to each other via the electrical conductor 23. When an external source is connected to the second connection element 22, the data processing system 31 and/or the power supply system 32 is supplied by the external source via the contacting of the first connection element 21 with the interface 33. Connecting an external source enables the feeding and reading of data sets such as control parameters and/or the charging of an integrated energy storage device to provide the necessary electrical energy.

FIG. 3 shows possible configurations of a connection part 4 according to the invention with an inner side 41 facing the prosthetic component 3 and an outer side 42 facing away from the prosthetic component. A first electrical connection element 21 is arranged on the inner side 41, and a second electrical connection element 22 is arranged on the outer side 42. The connection elements 21, 22 are electrically connected to each other.

A duct 43 is introduced into the connection part 4, for example by milling, drilling, casting, screwing on, gluing on or by other suitable means. The electrical conductor 23 is placed and secured in the duct 43 after production of the connection part 4, so that a prosthetic component 3 arranged in the cavity 12 of the base body 11 cannot become entangled in or chafe against the conductor 23.

In a further embodiment, the electrical conductor 23 is arranged in the connection part 4. Depending on the manufacturing method, this can be done during or after completion of the production of the connection part 4. The electrical conductor 23 may be molded or incorporated during an injection molding process or an additive manufacturing process.

In FIG. 4, a variant of the invention is shown which corresponds substantially to FIG. 1, but only the base body 11 is shown in a sectional view. In contrast to FIG. 1, in FIG. 4 the connection part 4 is arranged on the inner side of the base body 11. In the illustrated embodiment, the connection part 4 is attached to a proximal edge of the prosthetic component 3. The attachment may be detachable, for example by a clamp connection, by at least one fastening element, by Velcro fasteners, bolts, hooks, sliders or the like. The prosthetic cosmetic cover 1 is arranged around the prosthetic component 3 on the outer side, wherein the former can be fixed in various ways to the prosthetic component 3, which in the illustrated embodiment is formed as a part of a prosthetic knee joint. Here, too, the fastening is advantageously carried out such that it can be repeatedly detached, but a permanent fastening, for example by means of material bonding or permanent bonding, may also be provided. The cavity 12 within the prosthetic cosmetic cover 1 is almost completely filled by the prosthetic component 3, and a free space for inserting a lower leg tube can still be seen at the distal end. In this cavity 12, the inner side 13 can be seen, which faces the prosthetic component 3. The connection part 4 has its inner side facing the prosthetic component 3 and its outer side points away from the prosthetic component 3. The outer side is at least partially covered by the base body 11 of the prosthetic cosmetic cover 1, leaving the second electrical connection element 22 accessible from the outside. The connection part 4 is formed of multiple parts, and the multiple parts of the connection part 4 can be attached to one another, for example by means of a Velcro fastener or by means of form-fitting elements, such that they can be detached.

### REFERENCE LIST

1—Prosthetic cosmetic cover

11—Base body

12—Cavity

13—Inner side

14—Outer side

21—First electrical connection element

22—Second electrical connection element

23—Electrical conductor

3—Prosthetic component

31—Data processing system

32—Power supply system

33—Interface

4—Connection part

41—Inner side

42—Outer side

43—Duct

1. A prosthetic cosmetic cover, comprising:
a base body which forms a cavity for at least partially receiving a prosthetic component, wherein the base body comprises an inner side configured for facing a prosthetic component and an outer side configured for facing away from the prosthetic component,
at least one first electrical connection element arranged on the inner side of the base body, and
at least one second electrical connection element arranged on the outer side of the base body, wherein the at least one first electrical connection element and the at least one second electrical connection element are electrically connected to one another.

2. The prosthetic cosmetic cover according to claim 1, further comprising an electrical conductor for connecting the at least one first electrical connection element and the at least one second electrical connection element.

3. The prosthetic cosmetic cover according to claim 1, wherein the at least one first electrical connection element and the at least one second electrical connection element are designed as a socket, contact field, and/or plug.

4. The prosthetic cosmetic cover according to claim 1 further comprising a magnetic securing the at least one first electrical connection element and the at least one second electrical connection element.

5. The prosthetic cosmetic cover according to claim 1 wherein the base body is formed in multiple parts.

6. The prosthetic cosmetic cover according to claim 1 wherein the base body is formed at least partially from foam.

7. The prosthetic cosmetic cover according to claim 1 further comprising a connection part fastened to the base body, said connection part having an inner side facing the prosthetic component and an outer side facing away from the prosthetic component.

8. The prosthetic cosmetic cover according to claim 7, wherein the connection part is formed from a material that is more rigid than a remaining material of the base body.

9. The prosthetic cosmetic cover according to claim 1 wherein the at least one first electrical connection element is arranged on the inner side of the connection part and/or the at least one second electrical connection element is arranged on the outer side of the connection part.

10. The prosthetic cosmetic cover according to claim 7 further comprising an electrical conductor for connecting the at least one first electrical connection element and the at least one second electrical connection element, wherein the electrical conductor is arranged in the connection part.

11. The prosthetic cosmetic cover according to claim 7 wherein the at least one first electrical connection element is attached to the connection part in a form-fitting manner.

12. The prosthetic cosmetic cover according to claim 7 wherein the connection part is fixed to the base body in a form-fitting or material-bonded manner

13. The prosthetic cosmetic cover according to claim 7 wherein the connection part is formed in multiple parts.

14. The prosthetic cosmetic cover according to claim 7 wherein the connection part has at least one fastening element for fixing to the prosthetic component.

15. The prosthetic cosmetic cover according to claim 7 wherein the connection part is removably fastened to the base body.

16. The prosthetic cosmetic cover according to claim 7 wherein the connection part is arranged on the inner side of the base body.

17. The prosthetic cosmetic cover according to claim 2 wherein the electrical conductor is arranged in a duct in the base body.

18. A system comprising:
a prosthetic component; and
a prosthetic cosmetic cover according to claim 1, wherein the prosthetic cosmetic cover is attached to the prosthetic component in an applied state.

19. A system according to claim 18, further comprising
a data processing system and/or a power supply system arranged in the prosthetic component; and
at least one connection element connected to the data processing system and/or the power supply system.

20. A system according to claim 19, further comprising
an electrical interface arranged on the data processing system and/or the power supply system; and (32) and is connected to the
at least one connection element, wherein the electrical interface is connected to the at least one connection element and is electrically conductive.

21. A system according to claim 18 wherein the prosthetic cosmetic cover is detachably arranged on the prosthetic component.

22. A connection part configured as part of a multi-part base body of a prosthetic cosmetic cover according to claim 1, wherein the connection part comprises an inner side configured to face a prosthetic component and an outer side configured to face (away from the prosthetic component, wherein the connection part further comprises at least one first electrical connection element arranged on the inner side and at least one second electrical connection element arranged on the outer side, wherein the at least one first electrical connection element and the at least one second electrical connection element are electrically connected to one another.
